(19) United States
(12) Patent Application Publication
(10) Pub. No.: US 2026/0022090 A1
(43) Pub. Date: Jan. 22, 2026

(54) REMOVAL OF ALDEHYDES IN ACETIC ACID PRODUCTION

(71) Applicant: LyondellBasell Acetyls, LLC, Houston, TX (US)

(72) Inventors: Shane J. Weber, League City, TX (US); Noel C. Hallinan, Loveland, OH (US); Ryan J. Mathews, Tomball, TX (US); David L. Ramage, Friendswood, TX (US)

(73) Assignee: LyondellBasell Acetyls, LLC, Houston, TX (US)

(21) Appl. No.: 19/271,012

(22) Filed: Jul. 16, 2025

Related U.S. Application Data
(60) Provisional application No. 63/673,261, filed on Jul. 19, 2024.

(51) Int. Cl. C07C 51/145; C07C 51/46; C07C 51/50

(52) U.S. Cl. CPC C07C 51/145; C07C 51/46; C07C 51/50

(57) ABSTRACT

Disclosed is a process and production system for producing acetic acid including steps for removal of acetaldehyde from the process. The steps for removal of acetaldehyde from the process include distillation, absorption, and stripping to concentrate acetaldehyde in a first light ends stream. Butane is added to the first light ends stream to form a second light ends stream comprising an azeotrope of butane and acetaldehyde. A method for removing acetaldehyde from a solution comprising acetaldehyde is also disclosed.

### PRIOR RELATED APPLICATION

This application claims the benefit of priority to U.S. Provisional Application No. 63/673,261, filed on Jul. 19, 2024, which is incorporated here by reference in its entirety.

### FIELD OF THE INVENTION

This disclosure relates to the production of acetic acid. More particularly, the disclosure relates to removal of acetaldehyde in acetic acid production.

### BACKGROUND OF THE INVENTION

In the current acetic acid production process, a reaction mixture is withdrawn from a reactor and is separated in a flash tank into a liquid fraction and a vapor fraction comprising acetic acid generated during the carbonylation reaction. The liquid fraction may be recycled to the carbonylation reactor, and the vapor fraction is passed to a separations unit, which by way of example may be a light-ends distillation column. The light-ends distillation column separates a crude acetic acid product from other components. The crude acetic acid product is passed to a drying column to remove water and then is subjected to further separations to recover acetic acid.

One challenge facing the industry is the presence of aldehyde(s) in acetic acid production, which can be present in the feed and also form as an undesired byproduct of carbonylation reactions. Processes for removing aldehydes exist; however, there continues to be a need to improve upon, and provide alternatives to, current aldehyde removal processes.

### SUMMARY OF THE INVENTION

An aspect of the disclosure relates to a process for producing acetic acid in an acetic acid production system. The process comprises: a) reacting methanol and carbon monoxide in the presence of a carbonylation catalyst to form acetic acid and a reactor vapor byproduct in an acetic acid production reactor; b) flashing a reaction mixture discharged from the acetic acid production reactor into a vapor stream and a liquid stream, the first vapor stream comprising acetic acid, methanol, methyl acetate, methyl iodide, carbon monoxide, carbon dioxide, water, hydrogen iodide, and mixtures thereof; c) separating the first vapor stream by distillation in a first distillation column into: (1) a product side stream comprising acetic acid and water; (2) a first distillation bottoms stream; and (3) a first distillation overhead stream comprising acetaldehyde, water, carbon monoxide, carbon dioxide, methyl iodide, methyl acetate, methanol and acetic acid, and mixtures thereof; d) condensing the first distillation overhead stream to form one or more liquid phase compositions and a second vapor stream, wherein the second vapor stream comprises a majority of the carbon monoxide and carbon dioxide and a minor portion of the acetaldehyde, methyl iodide, water, and acetic acid; e) contacting one or more of the reactor vapor byproduct, the first distillation overhead stream, and the second vapor stream with acetic acid in an absorber vessel to produce an absorber overhead stream and an absorber bottoms stream, wherein the absorber bottoms stream comprises acetaldehyde, methyl iodide, water, and acetic acid; f) separating the absorber bottoms stream in a stripper vessel for produce a stripper overhead stream and a stripper bottoms stream, wherein the stripper overhead stream comprises methyl iodide, acetaldehyde, water, and acetic acid; g) distilling the stripper overhead stream in a second distillation column to produce a second distillation overhead stream and a second distillation bottoms stream, wherein the second distillation overhead stream comprises methyl iodide, acetaldehyde, and water, and the second distillation bottoms stream comprises water, acetic acid, and methyl iodide; h) cooling the second distillation overhead stream to form a first liquid stream; i) contacting a first portion of the first liquid stream with an amount butane sufficient to form an azeotrope of butane with at least 50% of the acetaldehyde in the first portion of the first liquid stream to form a second liquid stream, comprising acetic acid, water, methyl acetate, methyl iodide, acetaldehyde, and the azeotrope; and j) removing the azeotrope from the second liquid stream.

Another aspect of the disclosure relates to a method for removing acetaldehyde from an acetic acid production system. The method comprises: providing from the acetic acid production system a first solution, comprising acetic acid, water, methyl acetate, methyl iodide, and acetaldehyde, wherein the acetaldehyde is present in a first concentration based on the total weight of the solution; contacting the first solution with an amount butane sufficient to form an azeotrope of butane with at least 50% of the first concentration of acetaldehyde to form a treated solution, comprising acetic acid, water, methyl acetate, methyl iodide, acetaldehyde, and the azeotrope; and removing the azeotrope from the second solution.

Yet another aspect relates to an acetic acid production system. The system comprises: an acetic acid production reactor to react methanol and carbon monoxide in the presence of a carbonylation catalyst to form acetic acid and a vapor byproduct; a flash vessel that receives a reaction mixture comprising the acetic acid from the reactor; a first distillation column that receives a vapor stream from the flash vessel; a first decanter that receives a condensed first overhead stream from the distillation column; a chiller and a knock-out drum that receive a first portion of a first vapor stream from the first decanter, wherein condensed water and acetic acid are removed from the first vapor stream in the knock-out drum; an absorber that receives one of more of the vapor byproduct from the acetic acid production reactor, a second portion of the first vapor stream from the first decanter, and a second vapor stream from the knock-out drum; a stripper that that receives a bottoms stream from the absorber; a second distillation column that receives an overhead stream from the stripper; and a third distillation column that receives a portion of an overhead stream from the second distillation column.

The above paragraphs present a simplified summary of the presently disclosed subject matter in order to provide a basic understanding of some aspects thereof. The summary is not an exhaustive overview, nor is it intended to identify key or critical elements to delineate the scope of the subject matter claimed below. Its sole purpose is to present some concepts in a simplified form as a prelude to the more detailed description set forth below.

### BRIEF DESCRIPTION OF THE DRAWINGS

The claimed subject matter may be understood by reference to the following description taken in conjunction with the accompanying drawings, in which like reference numerals identify like elements, and in which:

FIG. 1 is a schematic of an exemplary acetic acid production system in accordance with embodiments of the present techniques; and

FIG. 1A is a schematic of a continuation of FIG. 1 in accordance with embodiments of the present techniques.

While the disclosed process and system are susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

A detailed description of embodiments of the disclosed process follows. However, it is to be understood that the described embodiments are merely exemplary of the process and that the process may be embodied in various and alternative forms of the described embodiments. Therefore, specific procedural, structural and functional details which are addressed in the embodiments described herein are not to be interpreted as limiting, but merely as a basis for the claims and as a representative basis for teaching one skilled in the art to variously employ the disclosed process.

The designation of groups of the Periodic Table of the Elements as used herein is in accordance with the current IUPAC convention. The expression “HAc” is used herein as an abbreviation for acetaldehyde. The expression “Mel” is used herein as an abbreviation for methyl iodide. The expression “HI” is used herein as an abbreviation for hydrogen iodide. The expression “acac” is used herein as an abbreviation for acetoacetate anion, i.e., H₃CC(═O)CH₂C(═O)O—. Unless specifically indicated otherwise, the expression “wt %” as used herein refers to the percentage by weight of a particular component in the referenced composition. With respect to all ranges disclosed herein, such ranges are intended to include any combination of the mentioned upper and lower limits even if the particular combination is not specifically listed.

Embodiments of the disclosed process and system involve the production of acetic acid by carbonylating methanol in a carbonylation reaction. The carbonylation reaction may be represented by: CH₃OH+CO→CH₃COOH

Embodiments of the disclosed process include: (a) obtaining HI in an acetic acid production system; and (b) continuously introducing a complexing agent into the system, wherein the complexing agent and HI interact to form a complex. The following description elaborates upon the disclosed process.

### Acetic Acid Production

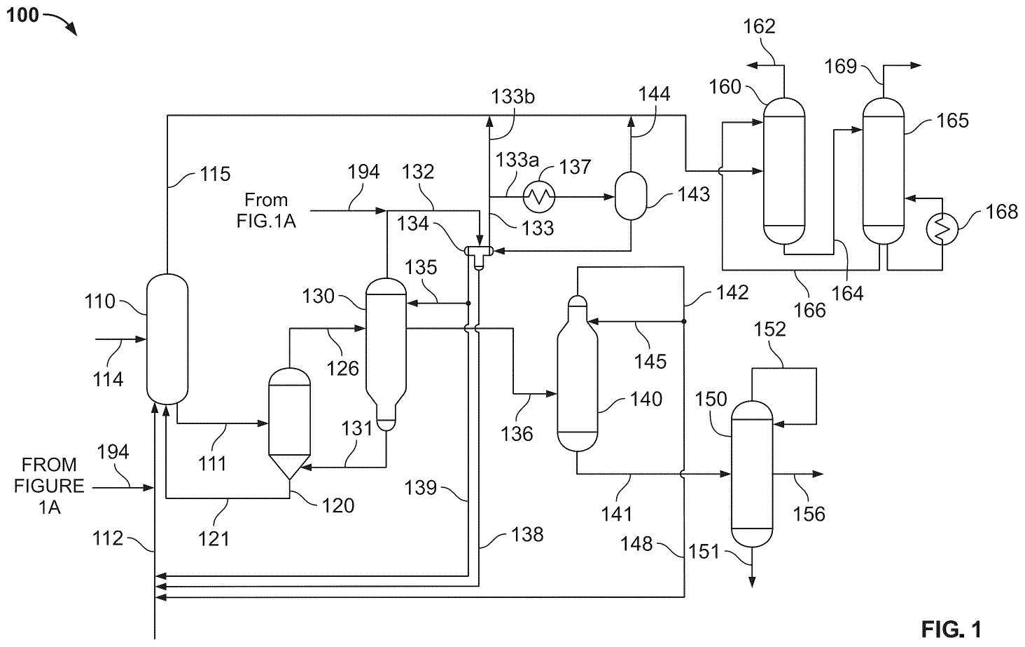

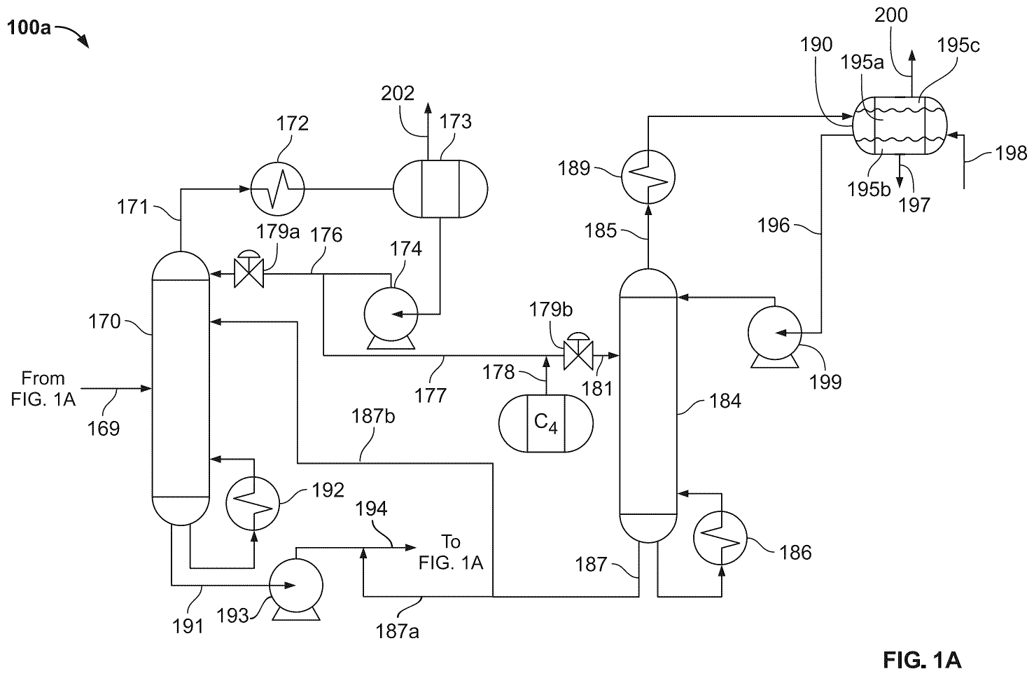

FIG. 1 is a schematic of an exemplary acetic acid production system 100 implementing the carbonylation reaction. In some embodiments, the acetic acid system 100 may include a reaction area 102, a light-ends area 104, and a purification area 106. The reaction area 102 may include a reactor 110, a flash vessel 120, and associated equipment. The reactor 110 is a reactor or vessel in which methanol is carbonylated in the presence of a catalyst to form acetic acid at elevated pressure and temperature.

The flash vessel 120 is a tank or vessel in which a reaction mixture obtained in the reactor is at least partially depressurized and/or cooled to form a vapor stream and a liquid stream. The liquid stream 121 may be a product or composition which has components in the liquid state under the conditions of the processing step in which the stream is formed. The vapor stream 126 may be a product or composition which has components in the gaseous state under the conditions of the processing step in which the stream is formed.

The light-ends area 104 may include a separations column, for example a light-ends column 130, and associated equipment such as decanter 134. The light-ends column is a fractioning or distillation column and includes equipment associated with the column, such as heat exchangers, decanters, pumps, compressors, valves, and the like. The purification area 106 may include a drying column 140, optionally a heavy-ends column 150, and associated equipment, and so on. The heavy-ends column is a fractioning or distillation column and includes any equipment associated with the column, such as heat exchangers, decanters, pumps, compressors, valves, and the like. Further, as discussed below, various recycle streams may include streams 121, 138, 139, and 148. The recycle streams may be products or compositions recovered from a processing step downstream of the flash vessel 120 and which is recycled to the reactor 110, flash vessel 120, or light-ends column 130, and so forth.

In an embodiment, the reactor 110 may be configured to receive a carbon monoxide feed stream 114 and a methanol feed stream 112. A reaction mixture may be withdrawn from the reactor in stream 111. Other streams may be included such as, for example, a stream that may recycle a bottoms mixture of the reactor 110 back into the reactor 110, or a stream may be included to release a gas from the reactor 110.

In an embodiment, the flash vessel 120 may be configured to receive stream 111 from the reactor 110. In the flash vessel 120, stream 111 may be separated into a vapor stream 126 and a liquid stream 121. The vapor stream 126 may be communicated to the light-ends column 130, and the liquid stream 121 may be communicated to the reactor 110. In an embodiment, stream 126 may have acetic acid, water, methyl iodide, methyl acetate, HI, mixtures thereof and the like.

In an embodiment, the light-ends column 130 may be a distillation column and associated equipment such as a decanter 134, pumps, compressors, valves, and other related equipment. The light-ends column 130 may be configured to receive stream 126 from the flash vessel 120. In the illustrated embodiment, stream 132 is the overhead product from the light-ends column 130, and stream 131 is bottoms product from the light-ends column 130. As indicated, light-ends column 130 may include a decanter 134, and stream 132 may pass into decanter 134.

Stream 135 may emit from decanter 134 and recycle back to the light-ends column 130. Stream 138 may emit from decanter 134 and may recycle back to the reactor 110 via, for example, stream 112 or be combined with any of the other streams that feed the reactor. Stream 139 may recycle a portion of the light phase of decanter 134 back to the reactor 110 via, for example, stream 112. Stream 136 may emit from the light-ends column 130. Other streams may be included such as, for example, a stream that may recycle a bottoms mixture of the light-ends column 130 back into the light-ends column 130. Streams received by or emitted from the light-ends column 130 may pass through a pump, compressor, heat exchanger, and the like as is common in the art.

In an embodiment, the drying column 140 may be a vessel and associated equipment such as heat exchangers, decanters, pumps, compressors, valves, and the like. The drying column 140 may be configured to receive stream 136 from the light-ends column 130. The drying column 140 may separate components of stream 136 into streams 142 and 141. Stream 142 may emit from the drying column 140, recycle back to the drying column via stream 145, and/or recycle back to the reactor 110 through stream 148 (via, for example, stream 112). Stream 141 may emit from the drying column 140 and may include de-watered crude acetic acid product. Stream 142 may pass through equipment such as, for example, a heat exchanger or separation vessel before streams 145 or 148 recycle components of stream 142. Other streams may be included such as, for example, a stream may recycle a bottoms mixture of the drying column 140 back into the drying column 140. Streams received by or emitted from the drying column 140 may pass through a pump, compressor, heat exchanger, separation vessel, and the like as is common in the art.

The heavy-ends column 150 may be a distillation column and associated equipment such as heat exchangers, decanters, pumps, compressors, valves, and the like. The heavy-ends column 150 may be configured to receive stream 141 from the drying column 140. The heavy-ends column 150 may separate components from stream 141 into streams 151, 152, and 156. Streams 151 and 152 may be sent to additional processing equipment (not shown) for further processing. Stream 152 may also be recycled, for example, to light-ends column 140. Stream 156 may have acetic acid product.

A single column (not depicted) may be used in the place of the combination of the light-ends distillation column 130 and the drying column 140. The single column may vary in the diameter/height ratio and the number of stages according to the composition of vapor stream from the flash separation and the requisite product quality. For instance, U.S. Pat. No. 5,416,237, the teachings of which are incorporated herein by reference, discloses a single column distillation. Alternative embodiments for the acetic acid production system 100 may also be found in U.S. Pat. Nos. 6,552,221, 7,524,988, and 8,076,512, which are herein incorporated by reference.

In an embodiment, the carbonylation reaction in reactor 110 of system 100 may be performed in the presence of a catalyst. Catalysts may include, for example, rhodium catalysts and iridium catalysts.

Suitable rhodium catalysts are taught, for example, by U.S. Pat. No. 5,817,869, which is herein incorporated by reference. The rhodium catalysts may include rhodium metal and rhodium compounds. In an embodiment, the rhodium compounds may be selected from the group consisting of rhodium salts, rhodium oxides, rhodium acetates, organo-rhodium compounds, coordination compounds of rhodium, the like, and mixtures thereof in an embodiment, the rhodium compounds may be selected from the group consisting of Rh₂(CO)₄I₂, Rh₂(CO)₄Br₂, Rh₂(CO)₄Cl₂, Rh(CH₃CO₂)₂, Rh(CH₃CO₂)₃, [H]Rh(CO)₂I₂, the like, and mixtures thereof. In an embodiment, the rhodium compounds may be selected from the group consisting of [H]Rh(CO)₂I₂, Rh(CH₃CO₂)₂, the like, and mixtures thereof.

Suitable iridium catalysts are taught, for example, by U.S. Pat. No. 5,932,764. The iridium catalysts may include iridium metal and iridium compounds. Examples of suitable iridium compounds include IrCl₃, IrI₃, IrBr₃, [Ir(CO)₂I]₂, [Ir(CO)₂Cl]₂, [Ir(CO)₂Br]₂, [Ir(CO)₄I₂]-H+, [Ir(CO)₂Br₂]-H+, [IR(CO)₂I₂]-H+, [Ir(CH₃)I₃(CO)₂]-H+, Ir₄(CO)I₂, IrCl₃·4H₂O, IrBr₃·4H₂O, Ir₃(CO)I₂, Ir₂O₃, IrO₂, Ir(acac)(CO)₂, Ir(acac)₃, Ir(OAc)₃, [Ir₃O(OAc)₆(H₂O)₃][OAc], H₂[IrCl₆], the like, and mixtures thereof. In an embodiment, the iridium compounds may be selected from the group consisting of acetates, oxalates, acetoacetates, the like, and mixtures thereof. In an embodiment, the iridium compounds may be one or more acetates.

In an embodiment, the catalyst may be used with a co-catalyst. In an embodiment, co-catalysts may include metals and metal compounds selected from the group consisting of osmium, rhenium, ruthenium, cadmium, mercury, zinc, gallium, indium, and tungsten, their compounds, the like, and mixtures thereof. In an embodiment, co-catalysts may be selected from the group consisting of ruthenium compounds and osmium compounds. In an embodiment, co-catalysts may be one or more ruthenium compounds. In an embodiment, the co-catalysts may be one or more acetates.

The reaction rate depends upon the concentration of the catalyst in the reaction mixture in reactor 110. In an embodiment, the catalyst concentration may be in a range from about 1.0 mmol to about 100 mmol catalyst per liter (mmol/l) of reaction mixture. In some embodiments the catalyst concentration is at least 2.0 mmol/l, or at least 5.0 mmol/l, or at least 7.5 mmol/l. In some embodiments the catalyst concentration is at most 75 mmol/l, or at most 50 mmol/l, or at least 25 mmol/l. In particular embodiments, the catalyst concentration is from about 2.0 to about 75 mmol/l, or from about 5.0 to about 50 mmol/l, or from about 7.5 to about 25 mmol/l.

In an embodiment, the carbonylation reaction in reactor 110 of system 100 may be performed in the presence of a catalyst stabilizer. Suitable catalyst stabilizers include at least two types of catalyst stabilizers. The first type of catalyst stabilizer may be a metal iodide salt such as lithium iodide. The second type of catalyst stabilizer may be a non-salt stabilizer. In an embodiment, non-salt stabilizers may be pentavalent Group VA oxides, such as that disclosed in U.S. Pat. No. 9,790,159 which is herein incorporated by reference. In an embodiment, the catalyst stabilizer may be one or more phosphine oxides. In an embodiment, the catalyst may be CYTOP™ 503 from Solvay.

The one or more phosphine oxides, in one or more embodiments, are represented by the formula R₃PO, where R is alkyl or aryl, O is oxygen, P is phosphorous. In one or more embodiments, the one or more phosphine oxides include a compound mixture of at least four phosphine oxides, where each phosphine oxide has the formula OPX₃, wherein O is oxygen, P is phosphorous and X is independently selected from C₄-C₁₈ alkyls, C₄-C₁₈ aryls, C₄-C₁₈ cyclic alkyls, C₄-C₁₈ cyclic aryls and combinations thereof. Each phosphine oxide has at least 15, or at least 18 total carbon atoms.

Examples of suitable phosphine oxides for use in the compound mixture include, but are not limited to, tri-n-hexylphosphine oxide (THPO), tri-n-octylphosphine oxide (TOPO), tris(2,4,4-trimethylpentyl)-phosphine oxide, tricyclohexylphosphine oxide, tri-n-dodecylphosphine oxide, tri-n-octadecylphosphine oxide, tris(2-ethylhexyl)phosphine oxide, di-n-octylethylphosphine oxide, di-n-hexylisobutylphosphine oxide, octyldiisobutylphosphine oxide, tribenzylphosphine oxide, di-n-hexylbenzylphosphine oxide, di-n-octylbenzylphosphine oxide, 9-octyl-9-phosphabicyclo [3.3.1]nonane-9-oxide, dihexylmonooctylphosphine oxide, dioctylmonohexylphosphine oxide, dihexylmonodecylphosphine oxide, didecylmonohexylphosphine oxide, dioctylmonodecylphosphine oxide, didecylmonooctylphosphine oxide, and dihexylmonobutylphosphine oxide and the like.

The compound mixture includes from 1 wt % to 60 wt %, or from 35 wt % to 50 wt % of each phosphine oxide based on the total weight of compound mixture. In one or more specific, non-limiting embodiments, the compound mixture includes TOPO, THPO, dihexylmonooctylphosphine oxide and dioctylmonohexylphosphine oxide. For example, the compound mixture may include from 40 wt % to 44 wt % dioctylmonohexylphosphine oxide, from 28 wt % to 32 wt % dihexylmonooctylphosphine oxide, from 8 wt % to 16 wt % THPO and from 12 wt % to 16 wt % TOPO, for example.

In one or more embodiments, the compound mixture exhibits a melting point of less than 20° C., or less than 10° C., or less than 0° C., for example.

In one or more specific embodiments, the compound mixture is Cyanex™ 923, commercially available from Cytec Corporation.

The amount of pentavalent Group VA oxide, when used, is such that a ratio to rhodium is greater than about 60:1. In some embodiments, the ratio of the pentavalent Group 15 oxide to rhodium is from about 60:1 to about 500:1. In some embodiments, from about 0.1 to about 3 M of the pentavalent Group 15 oxide may be in the reaction mixture. In some embodiments, from about 0.15 to about 1.5 M, or from 0.25 to 1.2 M, of the pentavalent Group 15 oxide may be in the reaction mixture.

In other embodiments, the reaction may occur in the absence of a stabilizer selected from the group of metal iodide salts and non-metal stabilizers such as pentavalent Group 15 oxides. In further embodiments, the catalyst stabilizer may consist of a complexing agent which is brought into contact with the reaction mixture stream 111 in the flash vessel 120.

In an embodiment, hydrogen may also be fed into the reactor 110. Addition of hydrogen can enhance the carbonylation efficiency. In an embodiment, the concentration of hydrogen may be in a range of from about 0.1 mol % to about 5 mol % of carbon monoxide in the reactor 110. In an embodiment, the concentration of hydrogen may be in a range of from about 0.3 mol % to about 3 mol % of carbon monoxide in the reactor 110.

In an embodiment, the carbonylation reaction in reactor 110 of system 100 may be performed in the presence of water. In an embodiment, the concentration of water is from about 2 wt % to about 14 wt % based on the total weight of the reaction mixture. In an embodiment, the water concentration is from about 2 wt % to about 10 wt %. In an embodiment, the water concentration is from about 4 wt % to about 8 wt %.

In an embodiment, the carbonylation reaction may be performed in the presence of methyl acetate. Methyl acetate may be formed in situ. In embodiments, methyl acetate may be added as a starting material to the reaction mixture. In an embodiment, the concentration of methyl acetate may be from about 2 wt % to about 20 wt % based on the total weight of the reaction mixture. In an embodiment, the concentration of methyl acetate may be from about 2 wt % to about 16 wt %. In an embodiment, the concentration of methyl acetate may be from about 2 wt % to about 8 wt %. Alternatively, methyl acetate or a mixture of methyl acetate and methanol from byproduct streams of the methanolysis of polyvinyl acetate or ethylene-vinyl acetate copolymers can be used for the carbonylation reaction.

In an embodiment, the carbonylation reaction may be performed in the presence of methyl iodide. Methyl iodide may be a catalyst promoter. In an embodiment, the concentration of Mel may be from about 0.6 wt % to about 36 wt % based on the total weight of the reaction mixture. In an embodiment, the concentration of Mel may be from about 4 wt % to about 24 wt %. In an embodiment, the concentration of Mel may be from about 6 wt % to about 20 wt %. Alternatively, Mel may be generated in the reactor 110 by adding HI.

In an embodiment, methanol and carbon monoxide may be fed to the reactor 110 in stream 112 and stream 114, respectively. The methanol feed stream to the reactor 110 may come from a syngas-methanol facility or any other source. Methanol does not react directly with carbon monoxide to form acetic acid. It is converted to Mel by the HI present in the reactor 110 and then reacts with carbon monoxide and water to give acetic acid and regenerate the HI.

In an embodiment, the carbonylation reaction in reactor 110 of system 100 may occur at a temperature within the range of about 120° C. to about 250° C., alternatively, about 150° C. to about 250° C., alternatively, about 150° C. to about 200° C. In an embodiment, the carbonylation reaction in reactor 110 of system 100 may be performed under a pressure within the range of about 200 psia (1.4 MPa-a) to 2000 psia (13.8 MPa-a), alternatively, about 200 psia (1,379 kPa-a) to about 1,000 psia (6.8 MPa-a), alternatively, about 300 psia (2.1 MPa-a) to about 500 psia (3.4 MPa-a).

In an embodiment, the reaction mixture may be withdrawn from the reactor 110 through stream 111 and is flashed in flash vessel 120 to form a vapor stream 126 and a liquid stream 121. The reaction mixture in stream 111 may include acetic acid, methanol, methyl acetate, methyl iodide, carbon monoxide, carbon dioxide, water, HI, heavy impurities, catalyst, or combinations thereof. The flash vessel 120 may comprise any configuration for separating vapor and liquid components via a reduction in pressure. For example, the flash vessel 120 may comprise a flash tank, nozzle, valve, or combinations thereof.

The flash vessel 120 may have a pressure below that of the reactor 110. In an embodiment, the flash vessel 120 may have a pressure of from about 10 psig (69 kPa-g) to 100 psig (690 kPa-g). In an embodiment, the flash vessel 120 may have a temperature of from about 100° C. to 160° C.

The vapor stream 126 may include acetic acid and other volatile components such as methanol, methyl acetate, methyl iodide, carbon monoxide, carbon dioxide, water, entrained HI, complexed HI, and mixtures thereof. The liquid stream 121 may include the catalyst, complexed HI, HI, an azeotrope of HI and water, and mixtures thereof. The liquid stream 121 may further comprise sufficient amounts of water and acetic acid to carry and stabilize the catalyst, non-volatile catalyst stabilizers, or combinations thereof. The liquid stream 121 may recycle to the reactor 110. The vapor stream 126 may be communicated to light-ends column 130 for distillation.

In an embodiment, the vapor stream 126 may be distilled in a light-ends column 130 to form an overhead stream 132, a crude acetic acid product stream 136, and a bottom stream 131. In an embodiment, the light-ends column 130 may have at least 10 theoretical stages or 16 actual stages. In an alternative embodiment, the light-ends column 130 may have at least 14 theoretical stages. In an alternative embodiment, the light-ends column 130 may have at least 18 theoretical stages. In embodiments, one actual stage may equal approximately 0.6 theoretical stages. Actual stages can be trays or packing. The reaction mixture may be fed via stream 126 to the light-ends column 130 at the bottom or the first stage of the column 130.

Stream 132 may include acetaldehyde, water, carbon monoxide, carbon dioxide, methyl iodide, methyl acetate, methanol and acetic acid, and mixtures thereof. Stream 131 may have acetic acid, methyl iodide, methyl acetate, HI, water, and mixtures thereof. Stream 136 may have acetic acid, HI, water, heavy impurities, and mixtures thereof.

In an embodiment, the light-ends column 130 may be operated at an overhead pressure within the range of 20 psia (138 kPa-a) to 40 psia (276 kPa-a), alternatively, the overhead pressure may be within the range of 30 psia (207 kPa-a) to 35 psia (241 kPa-a). In an embodiment, the overhead temperature may be within the range of 95° C. to 135° C., alternatively, the overhead temperature may be within the range of 110° C. to 135° C., alternatively, the overhead temperature may be within the range of 125° C. to 135° C. In an embodiment, the light-ends column 130 may be operated at a bottom pressure within the range of 25 psia (172 kPa-a) to 45 psia (310 kPa-a), alternatively, the bottom pressure may be within the range of 30 psia (207 kPa-a) to 40 psia (276 kPa-a).

In an embodiment, the bottom temperature may be within the range of 115° C. to 155° C., alternatively, the bottom temperature is within the range of 125° C. to 135° C. In an embodiment, crude acetic acid in stream 136 may be emitted from the light-ends column 140 as a liquid side-draw. Stream 136 may be operated at a pressure within the range of 25 psia (172 kPa-a) to 45 psia (310 kPa-a), alternatively, the pressure may be within the range of 30 psia (207 kPa-a) to 40 psia (276 kPa-a). In an embodiment, the temperature of stream 136 may be within the range of 110° C. to 140° C., alternatively, the temperature may be within the range of 125° C. to 135° C. Stream 136 may be taken between the fifth to the eighth actual stage of the light-ends column 130.

In one or more embodiments, the crude acetic acid in stream 136 may be optionally subjected to further purification, such as, but not limited to, drying-distillation, in drying column 140 to remove water and heavy-ends distillation in stream 141. Stream 141 may be communicated to heavy-ends column 150 where heavy impurities such as propionic acid may be removed in stream 151 and final acetic acid product may be recovered in stream 156.

The overhead stream 132 from the light-ends column 130 may be condensed and decanted in a decanter 134 to form one or more liquid phase compositions, such as a light aqueous phase and a heavy organic phase, and a vapor phase composition. In some embodiments, a portion or all of the vapor phase may be sent as stream 133b or 144 for further processing, as discussed below.

In some embodiments, the vapor phase composition emitted from the decanter 134 comprises gases (primarily CO and CO₂), methyl iodide, light alkanes, acetaldehyde, acetic acid, or a combination thereof, flows via stream 133 to chiller 137. As used herein, “light alkanes” refers to linear and/or branched alkanes having six or less carbon atoms. In some embodiments, the vapor phase stream 133 may have a water concentration of less than 50 wt %, less than 40 wt %, or less than 30 wt %. In some embodiments, stream 133 may have Mel greater than 25%, greater than 35%, or greater than 45% by weight of the stream. In some embodiments, stream 133a flows through chiller 137 and knockout drum 143 to form stream 144. A portion of higher boiling material is removed from stream 133a in knockout drum 143 and recycled to decanter 134. In some embodiments, vapor phase composition stream 144 may have a water concentration of less than 25 wt %, less than 15 wt %, or less than 5 wt %. In some embodiments, stream 144 may have methyl iodide greater than 30%, greater than 40%, or greater than 50% by weight of the stream. Make-up water may be introduced into the decanter 134 via a separate stream.

Streams 133 and/or 144 comprise a majority of the carbon monoxide and carbon dioxide from overhead stream 132. In some embodiments, a majority of the carbon monoxide and carbon dioxide means greater than or equal to 90 wt %, greater than or equal to 92 wt %, greater than or equal to 94 wt %, greater than or equal to 96 wt %, or greater than or equal to 98 wt %, of each carbon monoxide and carbon dioxide from overhead stream 132.

Streams 133 and/or 144 comprise a minor portion of the acetaldehyde, methyl iodide, water, and acetic acid from overhead stream 132. In some embodiments, a minor portion of the acetaldehyde, methyl iodide, water, and acetic acid means less than or equal to 25 wt %, less than or equal to 20 wt %, less than or equal to 15 wt %, less than or equal to 10 wt %, or less than or equal to 5 wt %, of each acetaldehyde, methyl iodide, water, and acetic acid from overhead stream 132.

Reactor vent stream 115, decanter vapor stream 133b, and/or knockout drum vapor stream 144 form a light ends feed stream to light ends absorber 160. In practice, light ends absorber 160 can be one or more absorber vessels, and reactor vent stream 115, decanter vapor stream 133b, and/or knockout drum vapor stream 144 can be routed individually or in any combination to the one or more absorber vessels represented as light ends absorber 160 in FIG. 1. The light ends feed stream enters proximate to the lower portion of light ends absorber 160 at the lower to flow upwards while acetic acid as an absorbent enters at an upper portion of light ends absorber 160 to flow downwards. One of ordinary skill in the art would know standard design parameters for light ends absorber 160, such as, but not limited to, vertical distance between light ends feed and acetic acid absorbent, vessel diameter, and vessel internals (e.g., baffles, trays, packing, and the like), to provide sufficient contact of the counterflowing light ends feed and acetic acid absorbent to remove all or a substantial portion of the Mel in the light ends feed stream. In some embodiments, the light ends absorber 160 may be operated at conditions sufficient to produce overhead stream 162 at a pressure within the range of 25 psia (172 kPa-g) to 45 psia (310 kPa-a), alternatively, the bottom pressure may be within the range of 30 psia (207 kPa-a) to 40 psia (276 kPa-a), and a temperature within the range of from 70° F. (21° C.) to 140° F. (60° C.), from 75° F. (24° C.) to 130° F. (54° C.), from 80° F. (27° C.) to 120° F. (49° C.).

In some embodiments, the light ends absorber overhead stream 162 comprises primarily CO, CO₂, and/or H₂, to be removed from the acetic acid production process 100. In some embodiments, stream 162 is further processed to recover one or more of the CO, CO₂, and/or H₂, as feed to other process units (e.g., methanol production), purged to a flare, or a combination thereof.

In some embodiments, the light ends absorber bottoms stream 164 comprises primarily acetic acid and recovered Mel to be routed to light ends stripper 165. Heat is supplied to light ends stripper 165 via reboiler 168 to separate light ends absorber bottoms stream 164 into a light ends stripper vapor stream 169 and a light ends stripper bottoms stream 166. Light ends stripper bottoms stream 166 comprises primarily acetic acid to be recycled as absorbent to light ends absorber 160. Light ends stripper vapor stream 169 is sent as feed to the HAc removal system 100a. In some embodiments, the light ends stripper 165 may be operated at conditions sufficient to produce overhead stream 169 at a pressure within the range of from 25 psia (172 kPa-a) to 65 psia (448 kPa-a) or from 30 psia (207 kPa-a) to 60 psia (414 kPa-a), and a temperature within the range of 100° C. to 170° C., alternatively, within the range of 105° C. to 160° C., alternatively, within the range of 110° C. to 150° C.

It should be noted that removal of the troublesome byproduct acetaldehyde from the acetic acid system 100 via physical or chemical techniques has occupied significant research time in the art for over a decade. This problematic byproduct and its aldehyde derivatives may unfortunately impact product purity. Acetaldehyde may also serve undesirably as a precursor to various hydrocarbons which impact decanter 134 heavy density, and as a precursor to higher alkyl iodides which may require expensive adsorption beds for their removal, for example.

### Acetaldehyde Removal System

FIG. 1A shows the acetaldehyde removal system 100a. In some embodiments, all or at least a portion of the light ends stripper vapor stream 169 is fed to a first HAc concentration column 170. In first HAc concentration column 170, acetaldehyde is separated from higher boiling components, such as, but not limited to methyl acetate, water, and acetic acid, and concentrated in overhead stream 171. In one example of a first HAc concentration column 170, the light ends stripper vapor stream 169 is distilled to form a vapor stream comprising over 50%, over 80%, or over 90%, of the acetaldehyde from the light ends stripper vapor stream 169 and a bottoms stream comprising over 50%, over 80%, or over 90%, of the methyl iodide, over 50%, over 80%, or over 90%, of the methyl acetate, over 50%, over 80%, or over 90%, of water, and over 50%, over 80%, or over 90%, of acetic acid, from the light ends stripper vapor stream 169.

The overhead temperature of the distillation in the HAc concentration column 170 is below about 80° C., 75° C., or 70° C. so that the vapor stream 171 comprises less than 20 wt %, less than 15 wt %, or less than 10 wt % of light alkanes. In some embodiments, HAc concentration column 170 has a number of theoretical stages in the range of from 10 to 20, from 12 to 18, or from 14 to 16. As used herein, “light alkanes” refers to linear and/or branched alkanes having six or less carbon atoms. In particular examples, the overhead vapor stream 171 temperature of the distillation is within the range of about 43° C. (boiling point of Mel) to about 80° C., about 43° C. to about 75° C., or about 43° C. to about 70° C. In particular examples, the overhead vapor stream 171 be operated at a pressure within the range of 15 psig (103 kPa-g) to 35 psig (241 kPa-g), alternatively, the pressure may be within the range of 20 psig (138 kPa-g) to 30 psig (207 kPa-g). Lowering the overhead temperature of the HAc concentration column 170 desirably increases the concentration of Mel in the bottoms stream 191 but may result in a larger fraction of the total acetaldehyde entering HAc concentration column 170 in the bottoms stream 191. Increasing the overhead temperature of the HAc concentration column 170 desirably increases the fraction of the total acetaldehyde entering HAc concentration column 170 in the overhead stream 171 but may increase the concentration of Mel in the overhead stream 171. According to some embodiments, the bottoms stream 191 is sent by pump 193 as a stream 194 to the acetic acid production reactor 110, to light ends column overhead stream 132, or a combination thereof. Heat input to HAc concentration column 170 is provided by reboiler 192. As shown in FIG. 1A, streams 176 and 177 are the same composition.

The vapor stream 171 is cooled by cooling water or other refrigerant in condenser 172 and the resulting liquid is collected in reflux drum 173. Pump 174 sends the liquid from reflux drum 173 to provide reflux for HAc concentration column 170 and feed for HAc removal column 184, as controlled by control valves 179a and 179b, respectively. Gas stream 202, including non-condensables and a minor amount of HAc, is removed from the process. Reducing the temperature of vapor stream 171 will reduce losses of HAc in gas stream 202. In some embodiments, the vapor stream 171 is reduced by condenser 172 such that the stream entering reflux drum 173 is at a temperature less than or equal to 20° C., 15° C., 10° C., or 5° C.

As discussed below, the present techniques provide for an azeotropic pathway which contains thermodynamically-controlled steps to separate acetaldehyde from other constituents of a feed stream. As explained below, the thermodynamically-controlled azeotrope of HAc and an azeotropic agent is stable to temperature and has a sufficiently low boiling point to be removed efficiently by distillation. Conditions, such as concentration of the azeotropic agent, can be tailored to facilitate rapid formation of the azeotrope of HAc and the azeotropic agent.

According to the present techniques, acetaldehyde may be removed from the acetaldehyde removal system 100a, and thereby from acetic acid production system 100, by adding butane 178, as an azeotropic agent, to the HAc distillation column overhead stream 177. In some embodiments, butane is added in an amount sufficient to form an azeotrope with at least 50%, at least 75%, at least 90%, or substantially all of the aldehyde in stream 177. In some embodiments, the contacting of stream 178 with stream 177 is controlled at a temperature in the range of from 25° C. to 120° C., from 30° C. to 100° C., or from 35° C. to 80° C.

The boiling points of selected components of the combined feed stream to HAc removal column 184, comprising added butane 178 and HAc distillation column overhead stream 177, are shown in Table 1, below.

TABLE 1

| Compound | Boiling Point (° C.) |
|---|---|
| Acetic Acid | 118 |
| Water | 100 |
| Methyl Acetate | 57 |
| Methyl Iodide | 42 |
| HAc | 21 |
| Butane | 0 |
| Butane/HAc Azeotrope | −7 |

In HAc removal column 184, the butane/HAc azeotrope is separated from higher boiling components, such as, but not limited to methyl iodide, methyl acetate, water, and acetic acid. In one example of an HAc removal column 184, the stream 181 is distilled to form a vapor overhead stream 185, comprising the butane/HAc azeotrope, and a bottoms stream 187, comprising Mel. HAc is removed from the system by concentration of the butane/HAc azeotrope in overhead stream 185 along with a portion of free HAc that may be in the feed to HAc removal column 184.

In some embodiments, the overhead temperature of the distillation in the HAc removal column 184 is about 45° C. to about 95° C., about 55° C. to about 85° C., or about 65° C. to about 75° C. In particular examples, the overhead vapor stream 185 can be operated at a pressure within the range of 115 psig (793 kPa-g) to 135 psig (931 kPa-g), alternatively, the pressure may be within the range of 120 psig (827 kPa-g) to 130 psig (896 kPa-g). In some embodiments, HAc removal column 184 has a number of theoretical stages in the range of from 15 to 25, from 17 to 23, or from 19 to 21. Lowering the overhead temperature of the HAc removal column 184 desirably assures that all aldehyde or substantially all of the butane/HAc azeotrope will be concentrated in the overhead stream 185. Increasing the overhead temperature of the HAc removal column 184 desirably decreases the amount of butane sent to bottoms stream 187. According to some embodiments, the heat input to HAc removal column 184 is provided by reboiler 186.

The bottoms stream 187 from HAc removal column 184 is added to the discharge of pump 193 as stream 187a to form stream 194, sent as stream 187b as reflux to HAc concentration column 170, or a combination thereof. In some embodiments, operation of system 100a, as disclosed herein, results in removal 85%, 90%, or 95% of the acetaldehyde entering system 100a.

The overhead stream 185 from HAc removal column 184 is cooled in condenser 189 after which it is sent to decanter 190. Water is also added to decanter 190 via stream 198. The mixture of cooled overhead stream 185 and water is allowed to stratify to form a light liquid (organic phase) layer 195a, a heavy liquid (aqueous phase) layer 195b, and a vapor phase 195c. Without wishing to be bound by any particular theory, it is believed that at least a portion of the butane/HAc azeotrope breaks down in decanter 190. Then, free butane preferentially stays in the light liquid (organic phase) layer 195a, as it is more soluble in the organic phase, and free acetaldehyde preferentially moves into the heavy liquid (aqueous phase) layer 195b, as it is more soluble in the aqueous phase. A portion of the bottom layer 195b comprising water and acetaldehyde is purged to waste via stream 197, makeup water is added via stream 198 to maintain the heavy liquid layer 195b. Vapor phase 195c, comprising non-condensables, is vented from decanter 190 via stream 200.

A portion of the heavy liquid layer 195b is withdrawn as stream 197 to waste disposition. In some embodiments, the heavy liquid layer 195b comprises: i) water, butane, and acetaldehyde in an amount greater than or equal to 99 wt %; ii) water in an amount greater than or equal to 50 wt %; and iii) butane in an amount less than or equal to 20 wt %, wherein the weight percentages are based on the weight of the heavy liquid layer 195b.

A portion of the light liquid layer 195a is withdrawn as stream 196 through pump 199 and added to HAc removal column 184 as reflux. In some embodiments, the light liquid layer 195a comprises:
i) butane and acetaldehyde in an amount greater than or equal to 99 wt %; and
ii) butane in an amount greater than or equal to 70 wt %;
wherein the weight percentages are based on the weight of the light liquid layer 195a.

In some embodiments, the operating temperature of decanter 190 is in the range of form about −10° C. to about 45° C., about −5° C. to about 40° C., or about 0° C. to about 35° C. In some embodiments, the operating pressure of decanter 190 is the range of from 115 psig (793 kPa-g) to 135 psig (931 kPa-g), alternatively, the pressure may be within the range of 120 psig (827 kPa-g) to 130 psig (896 kPa-g).

### Certain Embodiments

In some aspects, a process for producing acetic acid in an acetic acid production system comprises:
a) reacting methanol and carbon monoxide in the presence of a carbonylation catalyst to form acetic acid and a reactor vapor byproduct in an acetic acid production reactor;
b) flashing a reaction mixture discharged from the acetic acid production reactor into a vapor stream and a liquid stream, the first vapor stream comprising acetic acid, methanol, methyl acetate, methyl iodide, carbon monoxide, carbon dioxide, water, hydrogen iodide, and mixtures thereof;
c) separating the first vapor stream by distillation in a first distillation column into: (1) a product side stream comprising acetic acid and water; (2) a first distillation bottoms stream; and (3) a first distillation overhead stream comprising acetaldehyde, water, carbon monoxide, carbon dioxide, methyl iodide, methyl acetate, methanol and acetic acid, and mixtures thereof;
d) condensing the first distillation overhead stream to form one or more liquid phase compositions and a second vapor stream, wherein the second vapor stream comprises a majority of the carbon monoxide and carbon dioxide and a minor portion of the acetaldehyde, methyl iodide, water, and acetic acid;
e) contacting one or more of the reactor vapor byproduct, the first distillation overhead stream, and the second vapor stream with acetic acid in an absorber vessel to produce an absorber overhead stream and an absorber bottoms stream, wherein the absorber bottoms stream comprises acetaldehyde, methyl iodide, water, and acetic acid;
f) separating the absorber bottoms stream in a stripper vessel for produce a stripper overhead stream and a stripper bottoms stream, wherein the stripper overhead stream comprises methyl iodide, acetaldehyde, water, and acetic acid;
g) distilling the stripper overhead stream in a second distillation column to produce a second distillation overhead stream and a second distillation bottoms stream, wherein the second distillation overhead stream comprises methyl iodide, acetaldehyde, and water, and the second distillation bottoms stream comprises water, acetic acid, and methyl iodide;
h) cooling the second distillation overhead stream to form a first liquid stream;
i) contacting a first portion of the first liquid stream with an amount butane sufficient to form an azeotrope of butane with at least 50% of the acetaldehyde in the first portion of the first liquid stream to form a second liquid stream, comprising acetic acid, water, methyl acetate, methyl iodide, acetaldehyde, and the azeotrope; and
j) removing the azeotrope from the second liquid stream.

In some embodiments, the process for producing acetic acid is further characterized by one of more of:
a) the amount butane is sufficient to form an azeotrope of butane with substantially all of the acetaldehyde in the first portion of the first liquid stream;
b) the stripper bottoms stream comprises acetic acid, and the process further comprises routing the stripper bottoms stream to the absorber as an absorbent medium;
c) the process further comprises recycling the second distillation bottoms stream within the acetic acid production system, wherein in further embodiments, the second distillation bottoms stream is recycled to the acetic acid production reactor, the first distillation overhead stream, or a combination thereof;
d) the process further comprises routing a second portion of the first liquid stream to the second distillation column as reflux; and
e) removing the azeotrope from the second liquid stream comprises distilling the second liquid stream in a third distillation column to form a third distillation overhead stream and a third distillation bottoms stream, wherein the third distillation overhead stream comprises the azeotrope.

In any or all embodiments of the process including the third distillation overhead stream, further embodiments of the process for producing acetic acid further comprises:
a) cooling the third distillation overhead stream to form a third liquid stream;
b) contacting the third liquid stream with an amount of water to form a mixture;
c) allowing the mixture to stratify to form a light liquid layer and a heavy liquid layer; d) withdrawing a portion of the heavy liquid layer, wherein the heavy liquid layer comprises:
   i) water, butane, and acetaldehyde in an amount greater than or equal to 99 wt %;
   ii) water in an amount greater than or equal to 50 wt %; and
   iii) butane in an amount less than or equal to 20 wt %;
   wherein the weight percentages are based on the weight of the heavy liquid layer.

In any or all embodiments of the process including the third liquid layer, further embodiments of the process for producing acetic acid are characterized by one of more of:
a) the light liquid layer comprises:
   i) butane and acetaldehyde in an amount greater than or equal to 99 wt %; and
   ii) butane in an amount greater than or equal to 70 wt %;
   wherein the weight percentages are based on the weight of the light liquid layer; and
b) the process further comprises:
   i) withdrawing a portion of the light liquid layer; and
   ii) adding the withdrawn portion of the light liquid layer to the third distillation column as reflux.

In any or all embodiments of the process including the third distillation bottoms stream, in further embodiments of the process for producing acetic acid comprise routing the third distillation bottoms stream to the second distillation column as reflux.

In other aspects, a method for removing acetaldehyde from an acetic acid production system comprises:
a) providing from the acetic acid production system a first solution, comprising acetic acid, water, methyl acetate, methyl iodide, and acetaldehyde, wherein the acetaldehyde is present in a first concentration based on the total weight of the solution;
b) contacting the first solution with an amount butane sufficient to form an azeotrope of butane with at least 50% of the first concentration of acetaldehyde to form a treated solution, comprising acetic acid, water, methyl acetate, methyl iodide, acetaldehyde, and the azeotrope; and
c) removing the azeotrope from the second solution.

In some embodiments, the method for removing acetaldehyde from an acetic acid production system is further characterized by one of more of:
a) the amount butane is sufficient to form an azeotrope of butane with substantially all of the acetaldehyde in the first portion of the first liquid stream; and
b) removing the azeotrope from the second solution comprises distilling the treated solution to form a first overhead portion and a first bottoms portion, wherein the first overhead portion comprises the azeotrope.

In any or all embodiments of the method for removing acetaldehyde from an acetic acid production system including the first overhead portion, further embodiments of the method comprise:
a) cooling the first overhead portion to form a second solution;
b) contacting the second solution with an amount of water to form a mixture;
c) allowing the mixture to stratify to form a light liquid layer and a heavy liquid layer;
d) withdrawing a portion of the heavy liquid layer, wherein the heavy liquid layer comprises:
   i) water, butane, and acetaldehyde in an amount greater than or equal to 99 wt %;
   ii) water in an amount greater than or equal to 50 wt %; and
   iii) butane in an amount less than or equal to 20 wt %;
   wherein the weight percentages are based on the weight of the heavy liquid layer.

In any or all embodiments of the method for removing acetaldehyde from an acetic acid production system including the light liquid layer, further embodiments of the method comprise:
a) the light liquid layer comprises:
   i) butane and acetaldehyde in an amount greater than or equal to 99 wt %; and
   ii) butane in an amount greater than or equal to 70 wt %;
   wherein the weight percentages are based on the weight of the light liquid layer; and
b) the first overhead portion further comprises a portion of the light liquid layer.

In yet other aspects, an acetic acid production system comprises:
a) an acetic acid production reactor to react methanol and carbon monoxide in the presence of a carbonylation catalyst to form acetic acid and a vapor byproduct;
b) a flash vessel that receives a reaction mixture comprising the acetic acid from the reactor;
c) a first distillation column that receives a vapor stream from the flash vessel;
d) a first decanter that receives a condensed first overhead stream from the distillation column;
e) a chiller and a knock-out drum that receive a first portion of a first vapor stream from the first decanter, wherein condensed water and acetic acid are removed from the first vapor stream in the knock-out drum;
f) an absorber that receives one of more of the vapor byproduct from the acetic acid production reactor, a second portion of the first vapor stream from the first decanter, and a second vapor stream from the knock-out drum;
g) a stripper that that receives a bottoms stream from the absorber;
h) a second distillation column that receives an overhead stream from the stripper; and
i) a third distillation column that receives a portion of an overhead stream from the second distillation column.

In some embodiments, the acetic acid production system further comprises one of more of:
a) a second decanter that receives an overhead stream from the third distillation column; and
b) a pump that receives a bottoms stream from the second distillation column and discharges to the acetic acid production reactor, the first distillation column overhead stream, or a combination thereof.

Although the disclosed process and system have been described in detail, it should be understood that various changes, substitutions and alterations can be made herein without departing from the spirit and scope of the invention as defined by the appended claims. Moreover, the scope of the present application is not intended to be limited to the particular embodiments of the processes, machines, compositions, means, methods, and/or steps described in the specification. As one of the ordinary skill in the art will readily appreciate from the present disclosure, processes, machines, compositions, means, methods, and/or steps, presently existing or later to be developed that perform substantially the same function or achieve substantially the same result as the corresponding embodiments described herein, may be utilized according to the present invention. Accordingly, the appended claims are intended to include within their scope such processes, machines, compositions, means, methods, and/or steps.

### EXAMPLES

The following investigations and examples are intended to be illustrative only, and are not intended to be, nor should they be construed as, limiting the scope of the present invention in any way.

### Test Methods

In Example 1, an Aspen computer simulation (ASPEN Plus V12 steady-state simulation) of process streams and conditions was used to simulate an embodiment of the invention. The simulated process flow diagram (“PFD”) is shown in FIG. 1A. Flow rates in the examples are shown on a normalized parts-per-hundred (pph) by weight basis, wherein the feed rate (stream 169) into FIG. 1A is 100 parts by weight.

### Example 1

Example 1 demonstrates an embodiment wherein a light ends stripper overhead stream 169 is fed to HAc removal system 100a. An Aspen simulated process flow diagram (“PFD”) is represented by FIG. 1A. In this PFD, the HAc concentration column 170 operates with a bottoms temperature of 199° F. (93° C.) and pressure of 35 psig (241 kPa) and an overhead temperature of 152° F. (67° C.) and pressure of 35 psig (241 kPa). Process conditions and compositions for streams 169, 171, 176, 177, 178, 185, 187, 191, 196, 197, and 198 are shown in Table 2, below. Table 2 shows the calculated concentration of acetaldehyde (“HAc”), methyl iodide (“Mel”), light alkanes (“LA”), methyl acetate (“MeAc”), water (“H₂O”), n-butane (nC₄), and acetic acid (“GAA”) in each identified stream. The mass balance in Table 2 indicates 0.45 parts by weight (0.45 wt %×100 parts) of HAc entering HAc concentration column 170 in light ends stripper overhead stream 169 and 0.426 parts by weight (28.4 wt %×1.5 parts) of HAc exiting system 100a in the bottoms stream 197 from decanter 190. This indicates that about 95% of the incoming HAc is removed by system 100a.

TABLE 2

| | Stream | | | | | | | | | | | |
|---|---|---|---|---|---|---|---|---|---|---|---|---|
| Stream Attribute | 169 | 171 | 176 | 177 | 178 | 185 | 187 | 191 | 196 | 197 | 198 | 202 |
| Flow (100 part basis) | 100.0 | 213.3 | 200.6 | 11.3 | 0.1 | 11.2 | 10.5 | 87.2 | 10.4 | 1.5 | 0.7 | 1.5 |
| Temp. (° F.) | 255 | 152 | 40 | 40 | 100 | 154 | 262 | 199 | 100 | 100 | 100 | 40 |
| Temp. (° C.) | 124 | 67 | 4 | 4 | 38 | 68 | 128 | 93 | 38 | 38 | 38 | 4 |
| Pressure (psig) | 36 | 35 | 150 | 150 | 150 | 125 | 125 | 35 | 125 | 125 | 125 | 35 |
| Pressure (kPa-g) | 248 | 241 | 1,034 | 1,034 | 1,034 | 862 | 862 | 241 | 862 | 862 | 862 | 241 |
| HAc (wt %) | 0.45 | 3.72 | 3.84 | 3.84 | 0.00 | 16.62 | 0.15 | 0.00 | 13.9 | 28.4 | 0.00 | 1.28 |
| MeI (wt %) | 53.6 | 80.5 | 83.1 | 83.1 | 0.0 | 1.9 | 89.0 | 50.4 | 2.1 | 0.1 | 0.0 | 14.8 |
| LA (wt %) | 5.1 | 9.7 | 10.0 | 10.0 | 0.0 | 0.0 | 10.8 | 4.5 | 0.0 | 0.0 | 0.0 | 1.2 |
| MeAc (wt %) | 1.1 | 0.1 | 0.1 | 0.1 | 0.0 | 0.0 | 0.1 | 1.3 | 0.0 | 0.0 | 0.0 | 0.0 |
| H₂O (wt %) | 1.4 | 2.9 | 3.0 | 3.0 | 0.0 | 3.1 | 0.0 | 1.2 | 0.2 | 67.8 | 100.0 | 0.1 |
| nC₄ (wt %) | 0.0 | 0.0 | 0.0 | 0.0 | 100.0 | 78.2 | 0.0 | 0.0 | 83.8 | 3.4 | 0.0 | 0.0 |
| GAA (wt %) | 37.2 | 0.0 | 0.0 | 0.0 | 0.0 | 0.0 | 0.0 | 42.6 | 0.0 | 0.0 | 0.0 | 0.0 |

In Example 1, HAc concentration column 170 was simulated with 15 theoretical stages, and HAc removal column 184 was simulated with 20 theoretical stages. Normalized heat input to reboiler 192 was 4.16 MMBTU per 100 lb (96.7 MJ per kg) acetaldehyde removal. Normalized heat input to reboiler 186 is 0.70 MMBTU per 100 lb (16.3 MJ per kg) acetaldehyde removal. One of the ordinary skill in the art will readily determine actual column sizing based on this disclosure, a desired feed rate to HAc concentration column 170, and a desired acetaldehyde removal rate.

For the sake of brevity, only certain ranges are explicitly disclosed herein. However, in addition to recited ranges, any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

All documents and references cited herein, including testing procedures, publications, patents, journal articles, etc., are herein fully incorporated by reference for all jurisdictions in which such incorporation is permitted and to the extent such disclosure is consistent with the description of the present invention.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the processes, machines, means, processes, and/or steps described in the specification. As one of the ordinary skill in the art will readily appreciate from the disclosure of the present invention, processes, machines, means, processes, and/or steps, presently existing or later to be developed that perform substantially the same function or achieve substantially the same result as the corresponding embodiments described herein, may be utilized according to the present invention. Accordingly, the appended claims are intended to include within their scope such processes, machines, means, processes, and/or steps.

What is claimed is:

1. A process for producing acetic acid in an acetic acid production system, said process comprising:
a) reacting methanol and carbon monoxide in the presence of a carbonylation catalyst to form acetic acid and a reactor vapor byproduct in an acetic acid production reactor;
b) flashing a reaction mixture discharged from the acetic acid production reactor into a vapor stream and a liquid stream, the first vapor stream comprising acetic acid, methanol, methyl acetate, methyl iodide, carbon monoxide, carbon dioxide, water, hydrogen iodide, and mixtures thereof;
c) separating the first vapor stream by distillation in a first distillation column into: (1) a product side stream comprising acetic acid and water; (2) a first distillation bottoms stream; and (3) a first distillation overhead stream comprising acetaldehyde, water, carbon monoxide, carbon dioxide, methyl iodide, methyl acetate, methanol and acetic acid, and mixtures thereof;
d) condensing the first distillation overhead stream to form one or more liquid phase compositions and a second vapor stream, wherein the second vapor stream comprises a majority of the carbon monoxide and carbon dioxide and a minor portion of the acetaldehyde, methyl iodide, water, and acetic acid;
e) contacting one or more of the reactor vapor byproduct, the first distillation overhead stream, and the second vapor stream with acetic acid in an absorber vessel to produce an absorber overhead stream and an absorber bottoms stream, wherein the absorber bottoms stream comprises acetaldehyde, methyl iodide, water, and acetic acid;
f) separating the absorber bottoms stream in a stripper vessel for produce a stripper overhead stream and a stripper bottoms stream, wherein the stripper overhead stream comprises methyl iodide, acetaldehyde, water, and acetic acid;
g) distilling the stripper overhead stream in a second distillation column to produce a second distillation overhead stream and a second distillation bottoms stream, wherein the second distillation overhead stream comprises methyl iodide, acetaldehyde, and water, and the second distillation bottoms stream comprises water, acetic acid, and methyl iodide;
h) cooling the second distillation overhead stream to form a first liquid stream;
i) contacting a first portion of the first liquid stream with an amount butane sufficient to form an azeotrope of butane with at least 50% of the acetaldehyde in the first portion of the first liquid stream to form a second liquid stream, comprising acetic acid, water, methyl acetate, methyl iodide, acetaldehyde, and the azeotrope; and
j) removing the azeotrope from the second liquid stream.

2. The process of claim 1, wherein the amount butane is sufficient to form an azeotrope of butane with substantially all of the acetaldehyde in the first portion of the first liquid stream.

3. The process of claim 1, wherein the stripper bottoms stream comprises acetic acid, and the process further comprises routing the stripper bottoms stream to the absorber as an absorbent medium.

4. The process of claim 1, further comprising recycling the second distillation bottoms stream within the acetic acid production system.

5. The process of claim 4, wherein the second distillation bottoms stream is recycled to the acetic acid production reactor, the first distillation overhead stream, or a combination thereof.

6. The process of claim 1, further comprising routing a second portion of the first liquid stream to the second distillation column as reflux.

7. The process of claim 1, wherein removing comprises distilling the second liquid stream in a third distillation column to form a third distillation overhead stream and a third distillation bottoms stream, wherein the third distillation overhead stream comprises the azeotrope.

8. The process of claim 7, further comprising:
cooling the third distillation overhead stream to form a third liquid stream;
contacting the third liquid stream with an amount of water to form a mixture;
allowing the mixture to stratify to form a light liquid layer and a heavy liquid layer;
withdrawing a portion of the heavy liquid layer, wherein the heavy liquid layer comprises:
water, butane, and acetaldehyde in an amount greater than or equal to 99 wt %;
water in an amount greater than or equal to 50 wt %; and
butane in an amount less than or equal to 20 wt %;
wherein the weight percentages are based on the weight of the heavy liquid layer.

9. The process of claim 8, wherein the light liquid layer comprises:
butane and acetaldehyde in an amount greater than or equal to 99 wt %; and
butane in an amount greater than or equal to 70 wt %;
wherein the weight percentages are based on the weight of the light liquid layer.

10. The process of claim 8, further comprising:
withdrawing a portion of the light liquid layer; and
adding the withdrawn portion of the light liquid layer to the third distillation column as reflux.

11. The process of claim 7, further comprising routing the third distillation bottoms stream to the second distillation column as reflux.

12. A method for removing acetaldehyde from an acetic acid production system, comprising:
providing from the acetic acid production system a first solution, comprising acetic acid, water, methyl acetate, methyl iodide, and acetaldehyde, wherein the acetaldehyde is present in a first concentration based on the total weight of the solution;
contacting the first solution with an amount butane sufficient to form an azeotrope of butane with at least 50% of the first concentration of acetaldehyde to form a treated solution, comprising acetic acid, water, methyl acetate, methyl iodide, acetaldehyde, and the azeotrope; and
removing the azeotrope from the second solution.

13. The method of claim 12, wherein the amount butane is sufficient to form an azeotrope of butane with substantially all of the acetaldehyde in the first portion of the first liquid stream

14. The method of claim 12, wherein removing comprises distilling the treated solution to form a first overhead portion and a first bottoms portion, wherein the first overhead portion comprises the azeotrope.

15. The method of claim 14, further comprising:
cooling the first overhead portion to form a second solution;
contacting the second solution with an amount of water to form a mixture;
allowing the mixture to stratify to form a light liquid layer and a heavy liquid layer;
withdrawing a portion of the heavy liquid layer, wherein the heavy liquid layer comprises:
water, butane, and acetaldehyde in an amount greater than or equal to 99 wt %;
water in an amount greater than or equal to 50 wt %; and
butane in an amount less than or equal to 20 wt %;
wherein the weight percentages are based on the weight of the heavy liquid layer.

16. The method of claim 15, wherein the light liquid layer comprises:
butane and acetaldehyde in an amount greater than or equal to 99 wt %; and
butane in an amount greater than or equal to 70 wt %;
wherein the weight percentages are based on the weight of the light liquid layer.

17. The method of claim 15, wherein first overhead portion further comprises a portion of the light liquid layer.

18. An acetic acid production system comprising:
an acetic acid production reactor to react methanol and carbon monoxide in the presence of a carbonylation catalyst to form acetic acid and a vapor byproduct;
a flash vessel that receives a reaction mixture comprising the acetic acid from the reactor;
a first distillation column that receives a vapor stream from the flash vessel;
a first decanter that receives a condensed first overhead stream from the distillation column;
a chiller and a knock-out drum that receive a first portion of a first vapor stream from the first decanter, wherein condensed water and acetic acid are removed from the first vapor stream in the knock-out drum;
an absorber that receives one of more of the vapor byproduct from the acetic acid production reactor, a second portion of the first vapor stream from the first decanter, and a second vapor stream from the knock-out drum;
a stripper that that receives a bottoms stream from the absorber;
a second distillation column that receives an overhead stream from the stripper; and
a third distillation column that receives a portion of an overhead stream from the second distillation column.

19. The acetic acid production system of claim 18, further comprising a second decanter that receives an overhead stream from the third distillation column.

20. The acetic acid production system of claim 18, further comprising a pump that receives a bottoms stream from the second distillation column and discharges to the acetic acid production reactor, the first distillation column overhead stream, or a combination thereof.
